(19) United States
(12) Patent Application Publication
(10) Pub. No.: US 2024/0197584 A1
(43) Pub. Date: Jun. 20, 2024

(54) Oral Care Compositions

(71) Applicant: Colgate-Palmolive Company, New York, NY (US)

(72) Inventors: Shaoyi ZHANG, South Bound Brook, NJ (US); Suman CHOPRA, Monroe, NJ (US)

(73) Assignee: Colgate-Palmolive Company, New York, NY (US)

(21) Appl. No.: 18/544,591

(22) Filed: Dec. 19, 2023

Related U.S. Application Data
(60) Provisional application No. 63/476,067, filed on Dec. 19, 2022.

(51) Int. Cl. A61K 8/22; A61K 8/24; A61K 8/25; A61K 8/34; A61K 8/73; A61K 8/81; A61Q 11/00

(52) U.S. Cl. CPC A61K 8/22; A61K 8/24; A61K 8/25; A61K 8/342; A61K 8/345; A61K 8/73; A61K 8/8182; A61Q 11/00; A61K 2800/28; A61K 2800/48; A61K 2800/57

(57) ABSTRACT

Aspects of the disclosure are directed to oral care compositions. In accordance with an aspect of the invention, provided is an oral care composition having a whitening agent comprising a peroxide, a thickening system comprising two or more of: (i) a silica thickener, (ii) crosslinked polyvinyl pyrrolidone, or (iii) one of xanthan gum, cetyl alcohol, or a combination thereof; and an abrasive comprising calcium pyrophosphate; and from about 10 wt. % to 90 wt. % of water, wherein the oral care composition has a pH of about 4 to about 8, and all weight percentages are based on the total weight of the oral care composition.

### CROSS-REFERENCE TO RELATED PATENT APPLICATIONS

This application claims priority to U.S. Appl. No. 63/476,067, titled Oral Care Compositions and filed Dec. 19, 2022 the content of which is incorporated herein by reference in its entirety for all purposes.

### BACKGROUND

Oral care compositions, such as dentifrice compositions, can typically include one or more ingredients for maintaining the health of the oral cavity. Consumers have become aware of and desiring oral care compositions that also maintain the aesthetics of their teeth. It can be desirable to have dentifrice compositions with a relatively high level of peroxide of at least about 1 wt. % of the total weight of the oral care composition to achieve high whitening efficacy. However, it can be challenging to formulate dentifrice compositions with a relatively high level of peroxide because peroxide can be reactive with other dentifrice components, such as fluoride, metal ions, abrasives, etc.

Oral care compositions containing a relatively high level of peroxide have been particularly difficult to formulate with abrasives and non-insignificant amounts of water. One approach to the formulation challenges associated with incorporating sufficient peroxide in a dentifrice composition can be to separately package and/or apply a peroxide separate from the dentifrice composition. Unfortunately, consumer compliance can be lower when a peroxide must be applied separately from the dentifrice composition.

As such, there is a need for an improved oral care composition having enhance peroxide stability.

### BRIEF SUMMARY

This summary is intended merely to introduce a simplified summary of some aspects of one or more implementations of the present disclosure. Further areas of applicability of the present disclosure will become apparent from the detailed description provided hereinafter. This summary is not an extensive overview, nor is it intended to identify key or critical elements of the present teachings, nor to delineate the scope of the disclosure. Rather, its purpose is merely to present one or more concepts in simplified form as a prelude to the detailed description below.

Aspects of the invention are directed to oral care compositions. In accordance with an aspect of the invention, provided is an oral care composition including a whitening agent comprising a peroxide, a thickening system comprising two or more of: (i) a silica thickener, (ii) crosslinked polyvinyl pyrrolidone, or (iii) one of xanthan gum, cetyl alcohol, or a combination thereof; an abrasive comprising calcium pyrophosphate; and from about 5 to about 90 wt. % of water, wherein the oral care composition has a pH of about 4 to about 8, and all weight percentages are based on the total weight of the oral care composition.

According to another aspect of the invention, provided is an oral care composition including a whitening agent comprising a peroxide, a thickening system comprising two or more of: (i) a silica thickener, (ii) crosslinked polyvinyl pyrrolidone, or (iii) xanthan gum; an abrasive comprising calcium pyrophosphate; from about 1 to about 50 wt. % of a humectant; and from about 5 to about 90 wt. % of water, wherein the oral care composition has a pH of about 4 to about 8, and all weight percentages are based on the total weight of the oral care composition.

In accordance with a further aspect of the invention, provided is an oral care composition including a whitening agent comprising a peroxide, a thickening system comprising two or more of: (i) a silica thickener, (ii) crosslinked polyvinyl pyrrolidone, or (iii) cetyl alcohol; an anionic sulfate based surfactant; an abrasive comprising calcium pyrophosphate; from about 1 to about 50 wt. % of a humectant; and from about 5 to about 90 wt. % of water, wherein the oral care composition has a pH of about 4 to about 8, and all weight percentages are based on the total weight of the oral care composition.

### DETAILED DESCRIPTION

For illustrative purposes, the principles of the present invention are described by referencing various exemplary embodiments thereof. Although certain embodiments of the invention are specifically described herein, one of ordinary skill in the art will readily recognize that the same principles are equally applicable to, and can be employed in other compositions and methods. Before explaining the disclosed embodiments of the present invention in detail, it is to be understood that the invention is not limited in its application to the details of any particular embodiment disclosed herein. The terminology used herein is for the purpose of description and not of limitation.

As used herein and in the appended claims, the singular forms “a”, “an”, and “the” include plural references unless the context dictates otherwise. The singular form of any class of the ingredients refers not only to one chemical species within that class, but also to a mixture of those chemical species. The terms “a” (or “an”), “one or more” and “at least one” may be used interchangeably herein. The terms “comprising”, “including”, and “having” may be used interchangeably. The term “include” should be interpreted as “include, but are not limited to”. The term “including” should be interpreted as “including, but are not limited to”.

As used throughout, ranges are used as shorthand for describing each and every value that is within the range. Any value within the range can be selected as the terminus of the range. Thus, a range from 1-5, includes specifically 1, 2, 3, 4 and 5, as well as subranges such as 2-5, 3-5, 2-3, 2-4, 1-4, etc.

The term “about” when referring to a number means any number within a range of 10% of the number. For example, the phrase “about 2 wt. %” refers to a number between and including 1.8 wt. % and 2.2 wt. %.

All references cited herein are hereby incorporated by reference in their entireties. In the event of a conflict in a definition in the present disclosure and that of a cited reference, the present disclosure controls.

The abbreviations and symbols as used herein, unless indicated otherwise, take their ordinary meaning. The abbreviation “wt. %” means percent by weight with respect to the oral care composition. The symbol “°” refers to a degree, such as a temperature degree or a degree of an angle. The symbols “h”, “min”, “mL”, “nm”, and “um” refer to hour, minute, milliliter, nanometer, and micrometer, respectively. The abbreviation “UV-VIS” referring to a spectrometer or spectroscopy, means Ultraviolet-Visible. The abbreviation “rpm” means revolutions per minute.

When referring to chemical structures, and names, the symbols “C”, “H”, and “O” mean carbon, hydrogen, and oxygen, respectively. The symbols “—”, “═” and “≡” mean single bond, double bond, and triple bond, respectively.

“Volatile”, as used herein, means having a flash point of less than about 100° C. “Non-volatile”, as used herein, means having a flash point of greater than about 100° C.

Any member in a list of species that are used to exemplify or define a genus, may be mutually different from, or overlapping with, or a subset of, or equivalent to, or nearly the same as, or identical to, any other member of the list of species. Further, unless explicitly stated, such as when reciting a Markush group, the list of species that define or exemplify the genus is open, and it is given that other species may exist that define or exemplify the genus just as well as, or better than, any other species listed.

The phrases, “a mixture thereof,” “a combination thereof,” or a combination of two or more thereof” do not require that the mixture include all of A, B, C, D, E, and F (although all of A, B, C, D, E, and F may be included). Rather, it indicates that a mixture of any two or more of A, B, C, D, E, and F can be included. In other words, it is equivalent to the phrase “one or more elements selected from the group consisting of A, B, C, D, E, F, and a mixture of any two or more of A, B, C, D, E, and F.” Likewise, the term “a salt thereof” also relates to “salts thereof.” Thus, where the disclosure refers to “an element selected from the group consisting of A, B, C, D, E, F, a salt thereof, and a mixture thereof,” it indicates that that one or more of A, B, C, D, and F may be included, one or more of a salt of A, a salt of B, a salt of C, a salt of D, a salt of E, and a salt of F may be included, or a mixture of any two of A, B, C, D, E, F, a salt of A, a salt of B, a salt of C, a salt of D, a salt of E, and a salt of F may be included.

All components and elements positively set forth in this disclosure can be negatively excluded from the claims. In other words, the oral care compositions of the instant disclosure can be free or essentially free of all components and elements positively recited throughout the instant disclosure. In some instances, the oral care compositions of the present disclosure may be substantially free of non-incidental amounts of the ingredient(s) or compound(s) described herein. A non-incidental amount of an ingredient or compound is the amount of that ingredient or compound that is added into the oral care composition by itself. For example, an oral care composition may be substantially free of a non-incidental amount of an ingredient or compound, although such ingredient(s) or compound(s) may be present as part of a raw material that is included as a blend of two or more compounds.

Some of the various categories of components identified may overlap. In such cases where overlap may exist and the oral care composition includes both components (or the composition includes more than two components that overlap), an overlapping compound does not represent more than one component. For example, certain compounds may be characterized as both a fluoride ion source and a stannous ion source. If a particular claim to an oral care composition includes both a polyol and a humectant, glycerin will serve only as either a polyol or a humectant—not both.

For readability purposes, the chemical functional groups are in their adjective form; for each of the adjectives, the word “group” is assumed. For example, the adjective “alkyl” without a noun thereafter, should be read as “an alkyl group.”

Aspects of the invention are directed to oral care compositions having enhanced peroxide stability. For instance, certain embodiments of the oral care compositions disclosed herein can achieve significantly improved peroxide stability even when the peroxide is in the presence of a high water content, abrasive(s), and the oral care composition has a pH of 3 or more. Without being limited to any particular theory, it was unexpected discovered that oral care compositions having particular thickening systems comprising certain combinations of thickening agents enhance the peroxide stability of such oral care compositions even in the presence of a high water content, an abrasive, and at a pH of 3 or more.

In accordance with an aspect of the invention, provided is an oral care composition including a whitening agent comprising a peroxide, a thickening system comprising two or more of: (i) a silica thickener, (ii) a crosslinked polyvinyl pyrrolidone, or (iii) one of xanthan gum, cetyl alcohol, or a combination thereof; an abrasive comprising calcium pyrophosphate; and from about 5 to 90 wt. % of water, wherein the oral care composition has a pH of about 4 to about 8, and all weight percentages are based on the total weight of the oral care composition.

According to another aspect of the invention, provided is an oral care composition including a whitening agent comprising a peroxide, a thickening system comprising two or more of: (i) a silica thickener, (ii) a crosslinked polyvinyl pyrrolidone, or (iii) xanthan gum; an abrasive comprising calcium pyrophosphate; from about 1 to about 50 wt. % of a humectant; and from about 5 to 90 wt. % of water, wherein the oral care composition has a pH of about 4 to about 8, and all weight percentages are based on the total weight of the oral care composition.

In accordance with a further aspect of the invention, provided is an oral care composition including a whitening agent comprising a peroxide, a thickening system comprising two or more of: (i) a silica thickener, (ii) a crosslinked polyvinyl pyrrolidone, or (iii) cetyl alcohol; an anionic sulfate based surfactant; an abrasive comprising calcium pyrophosphate; from about 1 to about 50 wt. % of a humectant; and from about 5 to 90 wt. % of water, wherein the oral care composition has a pH of about 4 to about 8, and all weight percentages are based on the total weight of the oral care composition.

The oral care compositions typically have a pH of greater than 3 and, preferably, about 4 or more. For example, the oral care compositions may be formulated to have a pH of about 4 about 5, about 6, about 7, about 8, or any range therebetween. In some embodiments, the oral care compositions have a pH of about 4 to about 8, about 4 to about 7, about 4 to about 6.5; from about 4.5 to about 8, about 4.5 to about 7, about 4.5 to about 6.5; from about 5 to about 8, about 5 to about 7, about 5 to about 6.5; about 5.5 to about 8, about 5.5 to about 7, about 5.5 to about 6.5, or any range or subrange thereof.

The oral care compositions preferably achieve an enhanced level of peroxide stability, such that the oral care composition exhibits a loss of peroxide of about 30% or less over a 13-week period at 40° when sealed in a typical dentifrice tube. In some embodiments, the oral care composition has a loss of peroxide of about 25% or less, preferably about 20% or less, preferably about 17% or less, preferably about 14% or less, preferably about 12% or less, preferably about 10% or less, preferably about 8% or less, or preferably about 6% or less, over a 13-week period at 40° when sealed in a PBL recyclable dentifrice tube.

The oral care compositions may be in the form of a dentifrice, a toothpaste, a gel, and/or the like. Suitable components, such as those listed below, may be included or excluded from the formulations for the oral care compositions disclosed herein depending on the specific combination of other ingredients and/or the form of the oral care compositions. Additionally or alternatively, the oral care compositions may in some embodiments have a single phase, which contains that components and/or ingredients of the oral care composition. In other embodiments, the oral care composition may include two or more phases, such as two, three, four, or five phases.

Typically, the oral care compositions include one or more whitening agent(s) comprising a peroxide, preferably in an effective amount. The total amount of whitening agent(s) present in the oral care composition may be from about 1 to about 30 wt. %, based on the total weight of the oral care composition. For example, the whitening agent(s) may be present in the oral care composition in an amount from about 1 to about 30 wt. %, about 1 to about 26 wt. %, about 1 to about 22 wt. %, about 1 to about 19 wt. %, about 1 to about 16 wt. %, about 1 to about 13 wt. %, about 1 to about 10 wt. %, about 1 to about 8 wt. %, about 1 to about 6 wt. %, about 1 to about 5 wt. %, about 1 to about 4 wt. %, about 1 to about 3 wt. %, about 1 to about 2 wt. %; from about 2 to about 30 wt. %, about 2 to about 26 wt. %, about 2 to about 22 wt. %, about 2 to about 19 wt. %, about 2 to about 16 wt. %, about 2 to about 13 wt. %, about 2 to about 10 wt. %, about 2 to about 8 wt. %, about 2 to about 6 wt. %, about 2 to about 5 wt. %, about 2 to about 4 wt. %; from about 3 to about 30 wt. %, about 3 to about 26 wt. %, about 3 to about 22 wt. %, about 3 to about 19 wt. %, about 3 to about 16 wt. %, about 3 to about 13 wt. %, about 3 to about 10 wt. %, about 3 to about 8 wt. %, about 3 to about 6 wt. %; from about 5 to about 30 wt. %, about 5 to about 26 wt. %, about 5 to about 22 wt. %, about 5 to about 19 wt. %, about 5 to about 16 wt. %, about 5 to about 13 wt. %, about 5 to about 10 wt. %, about 5 to about 8 wt. %; from about 8 to about 30 wt. %, about 8 to about 26 wt. %, about 8 to about 22 wt. %, about 8 to about 19 wt. %, about 8 to about 16 wt. %, about 8 to about 13 wt. %; from about 11 to about 30 wt. %, about 11 to about 26 wt. %, about 11 to about 22 wt. %, about 11 to about 19 wt. %, about 11 to about 16 wt. %; from about 14 to about 30 wt. %, about 14 to about 26 wt. %, about 14 to about 22 wt. %, about 14 to about 19 wt. %; from about 18 to about 30 wt. %, about 18 to about 26 wt. %, about 18 to about 22 wt. %, or any range or subrange thereof, based on the total weight of the oral are composition.

The whitening agent(s) typically comprise a peroxide source, such as hydrogen peroxide, peroxides of alkali and alkaline earth metals, organic peroxy compounds, peroxy acids, salts thereof, or combinations of two or more thereof. The peroxide source may include peroxides of alkali and alkaline earth metals include lithium peroxide, potassium peroxide, sodium peroxide, magnesium peroxide, calcium peroxide, barium peroxide, and a combination of two or more thereof. Examples of organic peroxy compounds include carbamide peroxide (also known as urea hydrogen peroxide), glyceryl hydrogen peroxide, alkyl hydrogen peroxides, dialkyl peroxides, alkyl peroxy acids, peroxy esters, diacyl peroxides, benzoyl peroxide, monoperoxyphthalate, or a combination of two or more thereof. Peroxy acids and their salts include organic peroxy acids, such as alkyl peroxy acids, monoperoxyphthalate, and a combination thereof as well as inorganic peroxy acid salts, such as persulfate, dipersulfate, percarbonate, perphosphate, perborate and persilicate salts of alkali and/or alkaline earth metals, such as lithium, potassium, sodium, magnesium, calcium and barium, and mixtures thereof. In various embodiments, the peroxide compound comprises hydrogen peroxide, urea peroxide, sodium percarbonate, or a combination of two or more thereof.

The one or more peroxide(s) may be a non-complexed peroxide. The non-complexed peroxide may be chosen from one or more of the peroxides described above. In some preferred embodiments, the non-complexed peroxide comprises hydrogen peroxide.

The total amount of whitening agent may comprise from about 0.01 to about 10 wt. % of a non-complexed peroxide source, based on the total weight of the oral care composition. For example, the whitening agent may comprise a non-complexed peroxide source in an amount from about 0.01 to about 10 wt. %, about 0.01 to about 8 wt. %, about 0.01 to about 6 wt. %, about 0.01 to about 4 wt. %, about 0.01 to about 3 wt. %, about 0.01 to about 2 wt. %, about 0.01 to about 1 wt. %; from about 0.1 to about 10 wt. %, about 0.1 to about 8 wt. %, about 0.1 to about 6 wt. %, about 0.1 to about 4 wt. %, about 0.1 to about 3 wt. %, about 0.1 to about 2 wt. %, about 0.1 to about 1 wt. %; from about 0.5 to about 10 wt. %, about 0.5 to about 8 wt. %, about 0.5 to about 6 wt. %, about 0.5 to about 4 wt. %, about 0.5 to about 3 wt. %, about 0.5 to about 2 wt. %, about 0.5 to about 1 wt. %; from about 1 to about 10 wt. %, about 1 to about 8 wt. %, about 1 to about 6 wt. %, about 1 to about 4 wt. %, about 1 to about 3 wt. %, about 1 to about 2 wt. %; from about 2 to about 10 wt. %, about 2 to about 8 wt. %, about 2 to about 6 wt. %, about 2 to about 4 wt. %; from about 3 to about 10 wt. %, about 3 to about 8 wt. %, about 3 to about 6 wt. %, about 3 to about 4 wt. %; from about 4 to about 10 wt. %, about 4 to about 8 wt. %, about 4 to about 6 wt. %, or any range or subrange thereof, based on the total weight of the oral care composition.

In some embodiments, however, the whitening agent may comprise a peroxide complex. The peroxide complex may comprise a peroxide component and a porous cross-linked polymer. As referred to herein, a “peroxide component” is any oxidizing compound comprising a bivalent oxygen-oxygen group. Peroxide components include peroxides and hydroperoxides disclosed above, such as hydrogen peroxide, peroxides of alkali and alkaline earth metals, organic peroxy compounds, peroxy acids, salts thereof, and mixtures thereof. In various embodiments, the peroxide component comprises hydrogen peroxide, urea peroxide, sodium percarbonate and mixtures thereof. In one embodiment, the peroxide component comprises hydrogen peroxide. In one embodiment, the peroxide component consists essentially of hydrogen peroxide.

The whitening agent may comprise a peroxide complex, such as PVP complexed with hydrogen peroxide, cross-linked PVP complexed with hydrogen peroxide, silica complexed with hydrogen peroxide, tetrasodium pyrophosphate complexed with hydrogen peroxide, or a combination of two or more thereof. The peroxide complex may, also or alternatively, be selected from N-vinyl heterocyclic polymers. Preferably, the N-vinyl heterocyclic polymer is crosslinked and adsorbs, absorbs, complexes, or otherwise retains the peroxide component. The chemical and physical characteristics of the particulate hinder the release of the peroxide compound from the polymer particulates, and in some embodiments provides controlled release of the peroxide compound. The peroxide complex preferably comprises a peroxide component at a level of from about 0.1 to about 25 wt. %, from about 1 to about 25 wt. %, from about 5 to about 18 wt. %, based on the weight of the peroxide complex. In some preferred embodiments, the oral care composition comprises a complex of peroxide adsorbed to cross-linked polyvinylpyrrollidone.

In some embodiments, a non-peroxide whitening agent may be included in the oral care composition. Whitening agents among those useful herein include non-peroxy compounds, such as chlorine dioxide, chlorites and hypochlorites. Chlorites and hypochlorites include those of alkali and alkaline earth metals such as lithium, potassium, sodium, magnesium, calcium and barium. Non-peroxide whitening agents also include colorants, such as titanium dioxide and hydroxyapatite, pigments or dyes.

The oral care compositions comprise a thickening system comprising two or more thickening agents. In some embodiments, the thickening system may comprise 3 or more thickening agents, such as 3, 4, 5, or 6 thickening agents, including ranges and subranges formed therefrom. The thickening system generally comprises two or more thickening agents chosen from (i) a silica thickener, (ii) crosslinked polyvinyl pyrrolidone, or (iii) one of xanthan gum, cetyl alcohol, or a combination thereof. The two or more thickening agents may be selected from silica thickeners, crosslinked polyvinyl pyrrolidones, or from xanthan gums and/or cetyl alcohol. In some instances, the thickening system consists essentially of or consist of two or more thickening agents chosen from (i) a silica thickener, (ii) crosslinked polyvinyl pyrrolidone, or (iii) one of xanthan gum, cetyl alcohol, or a combination thereof. Preferably, the surfactant system comprises two or more thickening agents, with at least one thickening agent selected from one of the groups of (i) a silica thickener, (ii) crosslinked polyvinyl pyrrolidone, or (iii) one of xanthan gum, cetyl alcohol, or a combination thereof, and at least one other thickening agent selected form a different group of (i) a silica thickener, (ii) crosslinked polyvinyl pyrrolidone, or (iii) one of xanthan gum, cetyl alcohol, or a combination thereof.

The thickening system may be present in the oral care composition in an amount from about 0.5 to about 20 wt. %, based on the total weight of the oral care composition. In some embodiments, to total amount of thickening system in the oral care composition is from about 0.5 to about 17 wt. %, about 0.5 to about 15 wt. %, about 0.5 to about 13 wt. %, about 0.5 to about 11 wt. %, about 0.5 to about 9 wt. %, about 0.5 to about 7 wt. %, about 0.5 to about 5 wt. %; from about 1 to about 20 wt. %, about 1 to about 17 wt. %, about 1 to about 15 wt. %, about 1 to about 13 wt. %, about 1 to about 11 wt. %, about 1 to about 9 wt. %, about 1 to about 7 wt. %, about 1 to about 5 wt. %; from about 3 to about 20 wt. %, about 3 to about 17 wt. %, about 3 to about 15 wt. %, about 3 to about 13 wt. %, about 3 to about 11 wt. %, about 3 to about 9 wt. %, about 3 to about 7 wt. %; from about 5 to about 20 wt. %, about 5 to about 17 wt. %, about 5 to about 15 wt. %, about 5 to about 13 wt. %, about 5 to about 11 wt. %, about 5 to about 9 wt. %; from about 7 to about 20 wt. %, about 7 to about 17 wt. %, about 7 to about 15 wt. %, about 7 to about 13 wt. %; from about 10 to about 20 wt. %, about 10 to about 17 wt. %, about 10 to about 15 wt. %, about 10 to about 13 wt. %; from about 13 to about 20 wt. %, about 13 to about 17 wt. %; from about 16 to about 20 wt. %, or including ranges and subranges thereof, based on the total weight of the oral care composition.

The thickening system may comprise one or more silica thickener(s). Silica thickeners typically form a polymeric structure or gel in an aqueous media. The silica thickeners may be chosen from precipitated silica, fumed silica, hydrated silica, colloidal silica, a metal silicate, and combinations thereof. In some instances, the silica thickener(s) comprises fumed silica, colloidal silica, or a combination thereof. The silica thickener may include a metal silicate chosen from sodium silicate, potassium silicate, and a combination of two or more thereof. Additional examples of silica thickeners are disclosed in U.S. Pat. No. 6,342,205, which is incorporated herein by reference in its entirety for all purposes.

The oral care compositions may be formulated to include one or more silica thickener(s) in an amount from about 0.5 to about 10 wt. %, based on the total weight of the oral care composition. For example, the total amount of silica thickener(s) in the oral care composition may be from about 0.5 to about 8 wt. %, about 0.5 to about 6 wt. %, about 0.5 to about 5 wt. %, about 0.5 to about 4 wt. %, about 0.5 to about 3 wt. %, about 0.5 to about 2 wt. %; from about 1 to about 10 wt. %, about 1 to about 8 wt. %, about 1 to about 6 wt. %, about 1 to about 5 wt. %, about 1 to about 4 wt. %, about 1 to about 3 wt. %, about 1 to about 2 wt. %; from about 2 to about 10 wt. %, about 2 to about 8 wt. %, about 2 to about 6 wt. %, about 2 to about 5 wt. %, about 2 to about 4 wt. %, about 2 to about 3 wt. %; from about 3 to about 10 wt. %, about 3 to about 8 wt. %, about 3 to about 6 wt. %, about 3 to about 5 wt. %, about 3 to about 4 wt. %; from about 4 to about 10 wt. %, about 4 to about 8 wt. %, about 4 to about 6 wt. %, about 4 to about 5 wt. %; from about 6 to about 10 wt. %, about 6 to about 8 wt. %, about 7 to about 10 wt. %, about 8 to about 10 wt. %, or any range or subrange therefrom, based on the total weight of the oral care composition.

The thickening system may, in some instances, include one or more polyvinyl pyrrolidone(s). Polyvinyl pyrrolidone generally refers to a polymer containing vinylpyrrolidone (e.g., N-vinylpyrrolidone, N-vinyl-2-pyrrolidione, and N-vinyl-2-pyrrolidinone) as a monomeric unit. The monomeric unit may include a polar imide group, four non-polar methylene groups, and a non-polar methane group. The polyvinyl pyrrolidone may have an average molecular weight in the range 5,000 to 100,000 g/mol, preferably in the range 5,000 to 50,000 g/mol. The polyvinyl pyrrolidone may, in some embodiments, have an average molecular weight of ranging from about 8,000 to 12,000 g/mol (e.g., about 10,000 g/mol), from about 27,000 to about 33,000 g/mol (e.g., about 30,000 g/mol) and/or from about 37,000 to about 43,000 g/mol (e.g., about 40,000 g/mol). The polyvinyl pyrrolidone may be in the form of a hydrogen peroxide-polyvinyl pyrrolidone polymer complex. Examples of polyvinyl pyrrolidone complexes include those disclosed in U.S. Pat. No. 5,122,370, the contents of which are incorporated herein by reference for all purposes. In some embodiments, the polymer comprises crosslinked polyvinyl pyrrolidone (crosslinked PVP). In at least one embodiment, the polymer consists essentially of or consists of polyvinyl pyrrolidone.

The oral care compositions may include one or more polyvinyl pyrrolidone(s) in an amount from about 0.5 to about 20 wt. %, based on the total weight of the oral care composition. In some embodiments, the total amount of polyvinyl pyrrolidone(s) in the oral care composition is from about 0.5 to about 17 wt. %, about 0.5 to about 15 wt. %, about 0.5 to about 13 wt. %, about 0.5 to about 11 wt. %, about 0.5 to about 9 wt. %, about 0.5 to about 7 wt. %, about 0.5 to about 5 wt. %; from about 1 to about 20 wt. %, about 1 to about 17 wt. %, about 1 to about 15 wt. %, about 1 to about 13 wt. %, about 1 to about 11 wt. %, about 1 to about 9 wt. %, about 1 to about 7 wt. %, about 1 to about 5 wt. %; from about 3 to about 20 wt. %, about 3 to about 17 wt. %, about 3 to about 15 wt. %, about 3 to about 13 wt. %, about 3 to about 11 wt. %, about 3 to about 9 wt. %, about 3 to about 7 wt. %; from about 5 to about 20 wt. %, about 5 to about 17 wt. %, about 5 to about 15 wt. %, about 5 to about 13 wt. %, about 5 to about 11 wt. %, about 5 to about 9 wt. %; from about 7 to about 20 wt. %, about 7 to about 17 wt. %, about 7 to about 15 wt. %, about 7 to about 13 wt. %; from about 10 to about 20 wt. %, about 10 to about 17 wt. %, about 10 to about 15 wt. %, about 10 to about 13 wt. %; from about 13 to about 20 wt. %, about 13 to about 17 wt. %; from about 16 to about 20 wt. % or including ranges and subranges thereof, based on the total weight of the oral care composition.

The oral care composition may include one or more xanthan gum(s). The amount of xanthan gum in the oral are composition may be from about 0.05 to about 7 wt. %, based on the total weight of the oral care composition. For example, the total amount of xanthan gum(s) in the oral care composition may be from about 0.05 to about 7 wt. %, about 0.05 to about 5 wt. %, about 0.05 to about 4 wt. %, about 0.05 to about 3 wt. %, about 0.05 to about 2 wt. %, about 0.05 to about 1 wt. %, about 0.05 to about 0.8 wt. %, about 0.05 to about 0.5 wt. %; from about 0.1 to about 7 wt. %, about 0.1 to about 5 wt. %, about 0.1 to about 4 wt. %, about 0.1 to about 3 wt. %, about 0.1 to about 2 wt. %, about 0.1 to about 1 wt. %, about 0.1 to about 0.8 wt. %, about 0.1 to about 0.5 wt. %; from about 0.3 to about 7 wt. %, about 0.3 to about 5 wt. %, about 0.3 to about 4 wt. %, about 0.3 to about 3 wt. %, about 0.3 to about 2 wt. %, about 0.3 to about 1 wt. %, about 0.3 to about 0.8 wt. %, about 0.3 to about 0.5 wt. %; from about 0.5 to about 7 wt. %, about 0.5 to about 5 wt. %, about 0.5 to about 4 wt. %, about 0.5 to about 3 wt. %, about 0.5 to about 2 wt. %, about 0.5 to about 1 wt. %, about 0.5 to about 0.8 wt. %; from about 0.8 to about 7 wt. %, about 0.8 to about 5 wt. %, about 0.8 to about 4 wt. %, about 0.8 to about 3 wt. %, about 0.8 to about 2 wt. %, about 0.8 to about 1 wt. %; from about 1 to about 7 wt. %, about 1 to about 5 wt. %, about 1 to about 4 wt. %, about 1 to about 3 wt. %, about 1 to about 2 wt. %; from about 1.5 to about 7 wt. %, about 1.5 to about 5 wt. %, about 1.5 to about 4 wt. %, about 1.5 to about 3 wt. %; from about 2.5 to about 7 wt. %, about 2.5 to about 5 wt. %, about 2.5 to about 4 wt. %; from about 4 to about 7 wt. %, about 4 to about 5 wt. %; from about 5 to about 7 wt. %, or any range or subrange thereof, based on the total weight of the oral care composition.

The thickening system may include additional thickening agents, such as polysaccharides other than xanthan gum, acrylates, polymers, polyitaconates, acrylamides, and combinations of two or more thereof. Examples of polysaccharides other than xanthan gum include cellulose, carboxymethyl hydroxyethylcellulose, cellulose acetate propionate carboxylate, hydroxyethylcellulose, hydroxyethyl ethylcellulose, hydroxypropylcellulose, hydroxypropyl methylcellulose, methyl hydroxyethylcellulose, microcrystalline cellulose, sodium cellulose sulfate, carrageenan, or a combination of two more thereof.

Examples of acrylates that may be included in the oral care composition include, e.g., isobutyl acrylate, tert-butyl acrylate, 2-ethylhexyl acrylate, lauryl acrylate, lauryl/tridecyl acrylate, cetyl acrylate, stearyl acrylate, cyclohexyl acrylate, benzyl acrylate, isobornyl acrylate, 2-methoxyethyl acrylate, 2-ethoxyethyl acrylate, 2-ethoxyethoxyethyl acrylate, 2-phenoxyethyl acrylate, tetrahydrofurfuryl acrylate, 2-hydroxyethyl acrylate, 2-hydroxypropyl acrylate, 4-hydroxybutyl acrylate, dimethylaminoethyl acrylate, 1,4-butanediol acrylate, or a combination of two or more thereof.

The acrylate may be chosen from diacrylates. In some embodiments, the oral care composition includes a diacrylate chosen from 1,4-butanediol, 1,6-hexanediol, tetraethylene glycol, tripropylene glycol, ethoxylated bisphenol-A, and a combination of two or more thereof. Triacrylate monomers include trimethylol propane, ethoxylated, glyceryl propoxy, and pentaerythritol.

Acrylates further include methacrylates, such as methyl methacrylate, ethyl methacrylate, n-butyl methacrylate, isobutyl methacrylate, tert-butyl methacrylate, 2-ethylhexyl methacrylate, lauryl methacrylate, alkyl methacrylate, tridecyl methacrylate, stearyl methacrylate, cyclohexyl methacrylate, benzyl methacrylate, isobornyl methacrylate, 2-hydroxyethyl methacrylate, 2-hydroxypropyl methacrylate, dimethylaminoethyl methacrylate, diethylaminoethyl methacrylate, glycidyl methacrylate, tetrahydrofurfuryl methacrylate, allyl methacrylate, ethylene glycol methacrylate, triethylene glycol methacrylate, tetraethylene glycol methacrylate, 1,3-butyleneglycol methacrylate, 1,6-hexanediol methacrylate, trimethylopropane methacrylate, ethoxyethyl methacrylate, trifluoroethyl methacrylate, or a combination of two or more thereof.

Examples of acrylamides include, but are not limited to, acrylamide, methacrylamide and di(C₁-C₃₀) alkyl-acrylamides and -methacrylamides such as those of methyl, ethyl, propyl, butyl, pentyl, hexyl and the like. N-substituted acrylamides that may be suitable include N-ethylacrylamide, N-tert-butylacrylamide, N-tert-octylacrylamide, N-octylacrylamide, N-decylacrylamide, N-dodecylacrylamide and the corresponding N-substituted methacrylamides. Other N-substituted acrylamides include N-hydroxymethyl acrylamide, N-isopropylacrylamide, N-methylacrylamide, N,N′-methylenebisacrylamide, N-isobutoxymethylacrylamide, N,N-dimethylacrylamide, 2-acrylamido-2-methylpropanesulfonic acid, or a combination of two or more thereof.

The thickening system may, in some embodiments, include a fatty alcohol and/or a surfactant. The fatty alcohol(s) may be selected from decyl alcohol, undecyl alcohol, dodecyl alcohol, myristyl alcohol, lauryl alcohol, cetyl alcohol, stearyl alcohol, cetearyl alcohol (cetyl alcohol and stearyl alcohol), isostearyl alcohol, isocetyl alcohol, behenyl alcohol, linalool, oleyl alcohol, isotridecyl alcohol, myricyl alcohol, and a mixture of two or more thereof. In some cases, the surfactant system comprises cetyl alcohol, stearyl alcohol, cetearyl alcohol, lauryl alcohol, or a combination thereof.

The amount of fatty alcohol present in the oral care composition may be from about 0.05 to about 7 wt. %, based on the total weight of the oral care composition. For example, the total amount of fatty alcohol(s) in the oral care composition may be from about 0.05 to about 7 wt. %, about 0.05 to about 5 wt. %, about 0.05 to about 4 wt. %, about 0.05 to about 3 wt. %, about 0.05 to about 2 wt. %, about 0.05 to about 1 wt. %, about 0.05 to about 0.8 wt. %, about 0.05 to about 0.5 wt. %; from about 0.1 to about 7 wt. %, about 0.1 to about 5 wt. %, about 0.1 to about 4 wt. %, about 0.1 to about 3 wt. %, about 0.1 to about 2 wt. %, about 0.1 to about 1 wt. %, about 0.1 to about 0.8 wt. %, about 0.1 to about 0.5 wt. %; from about 0.3 to about 7 wt. %, about 0.3 to about 5 wt. %, about 0.3 to about 4 wt. %, about 0.3 to about 3 wt. %, about 0.3 to about 2 wt. %, about 0.3 to about 1 wt. %, about 0.3 to about 0.8 wt. %, about 0.3 to about 0.5 wt. %; from about 0.5 to about 7 wt. %, about 0.5 to about 5 wt. %, about 0.5 to about 4 wt. %, about 0.5 to about 3 wt. %, about 0.5 to about 2 wt. %, about 0.5 to about 1 wt. %, about 0.5 to about 0.8 wt. %; from about 0.8 to about 7 wt. %, about 0.8 to about 5 wt. %, about 0.8 to about 4 wt. %, about 0.8 to about 3 wt. %, about 0.8 to about 2 wt. %, about 0.8 to about 1 wt. %; from about 1 to about 7 wt. %, about 1 to about 5 wt. %, about 1 to about 4 wt. %, about 1 to about 3 wt. %, about 1 to about 2 wt. %; from about 1.5 to about 7 wt. %, about 1.5 to about 5 wt. %, about 1.5 to about 4 wt. %, about 1.5 to about 3 wt. %; from about 2.5 to about 7 wt. %, about 2.5 to about 5 wt. %, about 2.5 to about 4 wt. %; from about 4 to about 7 wt. %, about 4 to about 5 wt. %; from about 5 to about 7 wt. %, or any range or subrange thereof, based on the total weight of the oral care composition.

In some preferred embodiments, the thickening system comprises an anionic surfactant, such as a sodium lauryl sulfate and/or sodium lauryl ether sulfate, and a fatty alcohol, such as cetyl alcohol and stearyl alcohol.

The oral care compositions may include one or more surfactant(s) in various amounts. The oral care composition may comprise one or more anionic surfactant, one or more cationic surfactant, one or more nonionic surfactant, or a combination of two or more thereof. For example, the oral care composition may include one or more anionic surfactant(s) in an amount of about 0.5 to about 10 wt. %, based on the total weight of the oral care composition. In some instances, the anionic surfactant(s) is present in the oral care composition in an amount from about 0.5 to about 10 wt. %, about 0.5 to about 8 wt. %, about 0.5 to about 6 wt. %, about 0.5 to about 5 wt. %, about 0.5 to about 4 wt. %, about 0.5 to about 3 wt. %, about 0.5 to about 2 wt. %; from about 1 to about 10 wt. %, about 1 to about 8 wt. %, about 1 to about 6 wt. %, about 1 to about 5 wt. %, about 1 to about 4 wt. %, about 1 to about 3 wt. %, about 1 to about 2 wt. %; from about 2 to about 10 wt. %, about 2 to about 8 wt. %, about 2 to about 6 wt. %, about 2 to about 5 wt. %, about 2 to about 4 wt. %, about 2 to about 3 wt. %; from about 3 to about 10 wt. %, about 3 to about 8 wt. %, about 3 to about 6 wt. %, about 3 to about 5 wt. %, about 3 to about 4 wt. %; from about 5 to about 10 wt. %, about 5 to about 8 wt. %, about 5 to about 6 wt. %; from about 7 to about 10 wt. %, about 7 to about 8 wt. %, about 8 to about 10 wt. %, or any range or subrange thereof, based on the total weight of the oral care composition.

The anionic surfactant(s) may be selected from sulfate based anionic surfactants and non-sulfate based anionic surfactants. For example, the oral care composition may include an anionic surfactant chosen from ammonium lauryl sulfate, ammonium lauryl ether sulfate, sodium dodecyl sulfate, sodium coco-sulfate, ammonium coco-sulfate, and a combination of two or more thereof. In some preferred embodiments, the oral care composition comprises sodium lauryl sulfate, sodium lauryl ether sulfate, or a combination thereof. In further embodiments, one or more anionic surfactant is selected from sodium lauryl sulfate, sodium lauryl ether sulfate, sodium lauryl sarcosinate, sodium cocoyl methyl taurate, sodium monoglyceride sulfate, sodium cetaryl sulfate, potassium cocoyl glycinate, sodium lauryl phosphate, sodium lauryl lactylate, sodium lauryl sulfoacetate, sodium lauryl glutamate, sodium lauryl isethionate, sodium laureth carboxylate, sodium dodecyl benzenesulfonate, and a combination of two or more thereof.

The one or more anionic surfactant(s) may be selected from non-sulfate based anionic surfactants, such as sulfonated monoglycerides of fatty acids, acyl isethionates, acyl sarcosinates, acyl taurate, and a combination of two or more thereof. Non-limiting examples of acyl isethionates include sodium isethionate, sodium cocoyl isethionate, sodium lauroyl methyl isethionate, and sodium cocoyl methyl isethionate. Sulfonated monoglycerides of fatty acids include sodium coconut monoglyceride sulfonates and the like. Examples of acyl sarcosinates include potassium lauroyl sarcosinate, potassium cocoyl sarcosinate, sodium cocoyl sarcosinate, sodium lauroyl sarcosinate, sodium myristoyl sarcosinate, sodium oleoyl sarcosinate, sodium palmitoyl sarcosinate, ammonium lauroyl sarcosinate, and combinations of two or more thereof. Examples of acyl taurate surfactants include sodium cocoyl taurate, potassium cocoyl taurate, potassium methyl cocoyl taurate, sodium caproyl methyl taurate, sodium cocoyl taurate, sodium lauroyl taurate, sodium methyl cocoyl taurate (SMCT), sodium methyl lauroyl taurate, sodium methyl myristoyl taurate, sodium methyl oleoyl taurate, sodium methyl palmitoyl taurate, sodium methyl stearoyl taurate, and combinations of two or more thereof. In some embodiments, the oral care composition comprises sodium lauroyl methyl taurate (or sodium methyl lauroyl taurate), sodium methyl cocoyl taurate (SMCT), or a combination thereof.

The anionic surfactant(s) may have at least one acyl group, preferably, comprising a carbon chain of 8 to 21 carbons. In some instance, the alkyl group(s) of the anionic surfactant(s) comprise a carbon chain of 8 to 19 carbons, 8 to 17 carbons, 8 to 15 carbons, 8 to 13 carbons, 8 to 11 carbons; 9 to 21 carbons, 9 to 19 carbons, 9 to 17 carbons, 9 to 15 carbons, 9 to 13 carbons, 9 to 11 carbons; 11 to 21 carbons, 11 to 19 carbons, 11 to 17 carbons, 11 to 15 carbons, 11 to 13 carbons; 13 to 21 carbons, 13 to 19 carbons, 13 to 17 carbons, or any range or subrange thereof. The anionic surfactants disclosed herein may be incorporated in a salt form. The salt form of the anionic surfactants may have an alkali metal (e.g., sodium or potassium) and/or ammonium group.

Additionally or alternatively, the oral care composition may comprise one or more nonionic surfactant(s). The oral care composition may include nonionic surfactant(s) in an amount of about 0.5 to about 10 wt. %, based on the total weight of the oral care composition. In some instances, the nonionic surfactant(s) is present in the oral care composition in an amount from about 0.5 to about 10 wt. %, about 0.5 to about 8 wt. %, about 0.5 to about 6 wt. %, about 0.5 to about 5 wt. %, about 0.5 to about 4 wt. %, about 0.5 to about 3 wt. %, about 0.5 to about 2 wt. %; from about 1 to about 10 wt. %, about 1 to about 8 wt. %, about 1 to about 6 wt. %, about 1 to about 5 wt. %, about 1 to about 4 wt. %, about 1 to about 3 wt. %, about 1 to about 2 wt. %; from about 2 to about 10 wt. %, about 2 to about 8 wt. %, about 2 to about 6 wt. %, about 2 to about 5 wt. %, about 2 to about 4 wt. %, about 2 to about 3 wt. %; from about 3 to about 10 wt. %, about 3 to about 8 wt. %, about 3 to about 6 wt. %, about 3 to about 5 wt. %, about 3 to about 4 wt. %; from about 5 to about 10 wt. %, about 5 to about 8 wt. %, about 5 to about 6 wt. %; from about 7 to about 10 wt. %, about 7 to about 8 wt. %, about 8 to about 10 wt. %, or any range or subrange thereof, based on the total weight of the oral care composition.

The one or more nonionic surfactant(s) may be selected from glucosides, compounds produced by the condensation of alkylene oxide groups (hydrophilic in nature) with an organic hydrophobic compound, which may be aliphatic or alkylaromatic in nature. Examples of glucoside surfactants include decyl glucoside, stearyl glucoside, lauryl glucoside, coco-glucoside, cetearyl glucoside, decyl lauryl glucoside, lauroyl ethyl glucoside, myristoyl ethyl glucoside, oleoyl ethyl glucoside, or a combination of two or more thereof. In some embodiments, the surfactant system includes one or more nonionic surfactant(s) chosen from lauryl glucoside, lauroyl ethyl glucoside, myristoyl ethyl glucoside, oleoyl ethyl glucoside, and a combination of two or more thereof. Additionally or alternatively, the glucoside surfactant may be chosen from polyglucosides, such as alkylpolyglucosides.

Further examples of nonionic surfactants include poloxamers, polyethylene oxide condensates of alkyl phenols, products derived from the condensation of ethylene oxide with the reaction product of propylene oxide and ethylene diamine, ethylene oxide condensates of aliphatic alcohols, acids, and esters, long chain tertiary amine oxides, long chain tertiary phosphine oxides, long chain dialkyl sulfoxides and mixtures of such materials. Additional examples of nonionic surfactants include polyoxyethylene, polyoxyethylene sorbitan esters, polyoxyl n₄₀-₆₀ hydrogenated castor oil, fatty alcohol ethoxylates, polyethylene oxide condensates of alkyl phenols, products derived from the condensation of ethylene oxide with the reaction product of propylene oxide and ethylene diamine, ethylene oxide condensates of aliphatic alcohols, long chain tertiary amine oxides, long chain tertiary phosphine oxides, long chain dialkyl sulfoxides, or a combination of two or more thereof. In some instances, the nonionic surfactant comprises amine oxides, fatty acid amides, ethoxylated fatty alcohols, block copolymers of polyethylene glycol and polypropylene glycol, glycerol alkyl esters, polyoxyethytene glycol octylphenol ethers, sorbitan alkyl esters, polyoxyethylene glycol sorbitan alkyl esters, or a combination of two or more thereof.

In some embodiments, the oral care composition may comprise one or more cationic surfactant(s). The oral care composition may include cationic surfactant(s) in an amount of about 0.5 to about 10 wt. %, based on the total weight of the oral care composition. In some instances, the cationic surfactant(s) is present in the oral care composition in an amount from about 0.5 to about 10 wt. %, about 0.5 to about 8 wt. %, about 0.5 to about 6 wt. %, about 0.5 to about 5 wt. %, about 0.5 to about 4 wt. %, about 0.5 to about 3 wt. %, about 0.5 to about 2 wt. %; from about 1 to about 10 wt. %, about 1 to about 8 wt. %, about 1 to about 6 wt. %, about 1 to about 5 wt. %, about 1 to about 4 wt. %, about 1 to about 3 wt. %, about 1 to about 2 wt. %; from about 2 to about 10 wt. %, about 2 to about 8 wt. %, about 2 to about 6 wt. %, about 2 to about 5 wt. %, about 2 to about 4 wt. %, about 2 to about 3 wt. %; from about 3 to about 10 wt. %, about 3 to about 8 wt. %, about 3 to about 6 wt. %, about 3 to about 5 wt. %, about 3 to about 4 wt. %; from about 5 to about 10 wt. %, about 5 to about 8 wt. %, about 5 to about 6 wt. %; from about 7 to about 10 wt. %, about 7 to about 8 wt. %, about 8 to about 10 wt. %, or any range or subrange thereof, based on the total weight of the oral care composition.

Examples of cationic surfactants that may be present in the oral care composition include cetyl pyridinium chloride, coamidopropyl PG dimonium chloride phosphate (Phospholipid CDM), myristylamidopropyl PG dimonium chloride phosphate (Phospholipid PTM), stearamidopropyl PG dimonium chloride phosphate (Phospholipid SV), steapyrium chloride (Catemol WPC), and other suitable cationic materials. The cationic surfactants may be present in the form of a salt.

The oral care compositions typically include an abrasive comprising calcium pyrophosphate. The abrasive(s) may include one or more abrasives based on insoluble phosphate salts, such as those chosen from calcium pyrophosphate, dicalcium orthophosphate dihydrate, tricalcium phosphate, and calcium polymetaphosphate, and a combination of two or more thereof. In some embodiments, the abrasive is comprised primarily of calcium pyrophosphate and, in at least one embodiment, calcium pyrophosphate is the sole abrasive in the oral care composition. For example, the calcium pyrophosphate may comprise about 50 wt. % or more, about 60 wt. % or more, about 70 wt. % or more, about 80 wt. % or more, about 95 wt. % or more, about 98 wt. % or more, or about 99 wt. % or more of the total amount of the abrasive in the oral care composition.

The amount of abrasive(s) present in the oral are composition may be from about 1 to about 35 wt. %, based on the total weight of the oral care composition. In some instances, the total amount of abrasive(s) present in the oral care composition is from about 1 to about 35 wt. %, about 1 to about 35 wt. %, about 1 to about 31 wt. %, about 1 to about 27 wt. %, about 1 to about 23 wt. %, about 1 to about 19 wt. %, about 1 to about 17 wt. %, about 1 to about 15 wt. %, about 1 to about 13 wt. %; from about 4 to about 35 wt. %, about 4 to about 35 wt. %, about 4 to about 31 wt. %, about 4 to about 27 wt. %, about 4 to about 23 wt. %, about 4 to about 19 wt. %, about 4 to about 17 wt. %, about 4 to about 15 wt. %, about 4 to about 13 wt. %; from about 7 to about 35 wt. %, about 7 to about 35 wt. %, about 7 to about 31 wt. %, about 7 to about 27 wt. %, about 7 to about 23 wt. %, about 7 to about 19 wt. %, about 7 to about 17 wt. %, about 7 to about 15 wt. %, about 7 to about 13 wt. %; from about 10 to about 35 wt. %, about 10 to about 35 wt. %, about 10 to about 31 wt. %, about 10 to about 27 wt. %, about 10 to about 23 wt. %, about 10 to about 19 wt. %, about 10 to about 17 wt. %, about 10 to about 15 wt. %; from about 13 to about 35 wt. %, about 13 to about 35 wt. %, about 13 to about 31 wt. %, about 13 to about 27 wt. %, about 13 to about 23 wt. %, about 13 to about 19 wt. %, about 13 to about 17 wt. %, about 13 to about 15 wt. %; from about 15 to about 35 wt. %, about 15 to about 35 wt. %, about 15 to about 31 wt. %, about 15 to about 27 wt. %, about 15 to about 23 wt. %, about 15 to about 19 wt. %; from about 17 to about 35 wt. %, about 17 to about 35 wt. %, about 17 to about 31 wt. %, about 17 to about 27 wt. %, about 17 to about 23 wt. %; from about 19 to about 35 wt. %, about 19 to about 35 wt. %, about 19 to about 31 wt. %, about 19 to about 27 wt. %, about 19 to about 23 wt. %, or any range or subrange thereof, based on the total weight of the oral care composition.

The oral care compositions may include an anti-calculus agent. The amount of anti-calculus agent(s) present in the oral are composition may be from about 0.5 to about 10 wt. %, based on the total weight of the oral care composition. In some instances, the anti-calculus agent(s) is present in the oral care composition in an amount from about 0.5 to about 10 wt. %, about 0.5 to about 8 wt. %, about 0.5 to about 6 wt. %, about 0.5 to about 5 wt. %, about 0.5 to about 4 wt. %, about 0.5 to about 3 wt. %, about 0.5 to about 2 wt. %; from about 1 to about 10 wt. %, about 1 to about 8 wt. %, about 1 to about 6 wt. %, about 1 to about 5 wt. %, about 1 to about 4 wt. %, about 1 to about 3 wt. %, about 1 to about 2 wt. %; from about 2 to about 10 wt. %, about 2 to about 8 wt. %, about 2 to about 6 wt. %, about 2 to about 5 wt. %, about 2 to about 4 wt. %, about 2 to about 3 wt. %; from about 3 to about 10 wt. %, about 3 to about 8 wt. %, about 3 to about 6 wt. %, about 3 to about 5 wt. %, about 3 to about 4 wt. %; from about 5 to about 10 wt. %, about 5 to about 8 wt. %, about 5 to about 6 wt. %; from about 7 to about 10 wt. %, about 7 to about 8 wt. %, about 8 to about 10 wt. %, or any range or subrange thereof, based on the total weight of the oral care composition.

The anti-calculus agent(s) may be chosen from phosphates and polyphosphates (e.g., pyrophosphates), polyaminopropanesulfonic acid (AMPS), hexametaphosphate salts, zinc citrate trihydrate, polypeptides, polyolefin sulfonates, polyolefin phosphates, diphosphonates, and combinations of two or more thereof. Examples of anti-calculus agents include tetrasodium pyrophosphate, ammonium tripolyphosphate, hexametaphosphate salt, polycarboxylate polymers, polyvinyl methyl ether/maleic anhydride (PVME/MA) copolymers, or combinations of two or more thereof. In some embodiments, the oral care composition includes one or more alkali metal polyphosphate, e.g., as tetrasodium pyrophosphate.

The oral care compositions may include a fluoride ion source. The fluoride ion source may be present in an effective amount. In some cases, the fluoride ion source is present in the oral care composition in an amount of about 0.1 to about 7 wt. %, based on the total weight of the oral care composition. For example, the fluoride ion source may be present in an amount of about 0.1 to about 7 wt. %, about 0.1 to about 5 wt. %, about 0.1 to about 3 wt. %, about 0.1 to about 2 wt. %, about 0.1 to about 1 wt. %; from about 0.4 to about 7 wt. %, about 0.4 to about 5 wt. %, about 0.4 to about 3 wt. %, about 0.4 to about 2 wt. %, about 0.4 to about 1 wt. %; from about 0.8 to about 7 wt. %, about 0.8 to about 5 wt. %, about 0.8 to about 3 wt. %, about 0.8 to about 2 wt. %, about 0.8 to about 1.5 wt. %; from about 1.4 to about 7 wt. %, about 1.4 to about 5 wt. %, about 1.4 to about 3 wt. %; from about 2 to about 7 wt. %, about 2 to about 5 wt. %, about 2 to about 3 wt. %; from about 3 to about 7 wt. %, about 3 to about 5 wt. %, about 3 to about 4 wt. %; from about 4 to about 7 wt. %, about 4 to about 6 wt. %, about 4 to about 5 wt. %; from about 5 to about 7 wt. %, about 5 to about 6 wt. %, or any range or subrange thereof, based on the total weight of the oral care composition.

The fluoride ion source may be selected from soluble fluoride ion salts. For example, the fluoride ion source may comprise sodium fluoride, potassium fluoride, calcium fluoride, zinc fluoride, zinc ammonium fluoride, lithium fluoride, ammonium fluoride, stannous fluoride, stannous fluorozirconate, sodium monofluorophosphate, potassium monofluorophosphate, laurylamine hydrofluoride, diethylaminoethyloctoylamide hydrofluoride, didecyldimethylammonium fluoride, cetylpyridinium fluoride, dilaurylmorpholinium fluoride, sarcosine stannous fluoride, glycine potassium fluoride, glycine hydrofluoride, amine fluorides or a combination of two or more thereof. In some embodiments, the fluoride ion source comprises sodium fluoride, sodium monofluorophosphate, or a combination thereof. Additional examples of fluoride ion sources are disclosed in U.S. Pat. Nos. 3,535,421, 4,885,155, and 3,678,154, the disclosures of which are hereby incorporated by reference in their entirety for all purposes.

The oral care compositions may include a desensitizing agent. The amount of desensitizing agent may be from about 0.5 to about 9 wt. %, based on the total weight of the oral care composition. For example, the amount of desensitizing agent present in the oral care composition may be from about 0.5 to about 8 wt. %, about 0.5 to about 6 wt. %, about 0.5 to about 5 wt. %, about 0.5 to about 4 wt. %, about 0.5 to about 3 wt. %, about 0.5 to about 2 wt. %; from about 1 to about 9 wt. %, about 1 to about 8 wt. %, about 1 to about 6 wt. %, about 1 to about 5 wt. %, about 1 to about 4 wt. %, about 1 to about 3 wt. %, about 1 to about 2 wt. %; from about 2 to about 9 wt. %, about 2 to about 8 wt. %, about 2 to about 6 wt. %, about 2 to about 5 wt. %, about 2 to about 4 wt. %, about 2 to about 3 wt. %; from about 3 to about 9 wt. %, about 3 to about 8 wt. %, about 3 to about 6 wt. %, about 3 to about 5 wt. %, about 3 to about 4 wt. %; from about 4 to about 9 wt. %, about 4 to about 8 wt. %, about 4 to about 6 wt. %, about 4 to about 5 wt. %; from about 5 to about 9 wt. %, about 5 to about 8 wt. %, about 5 to about 6 wt. %; from about 6 to about 9 wt. %, about 6 to about 8 wt. %; from about 7 to about 9 wt. %, about 7 to about 8 wt. %, about 8 to about 10 wt. %, or any range or subrange therefrom, based on the total weight of the oral care composition.

The desensitizing agent(s) may comprise, e.g., benzocaine, potassium nitrate; potassium fluoride; strontium chloride; potassium chloride; potassium citrate; iron oxalate; sodium nitrate; lithium nitrate; magnesium nitrate; calcium nitrate; calcium hydroxide; dibasic calcium phosphate; strontium acetate; sodium monofluorophosphate; bisabolol; a local or systemic analgesic agent such as NSAIDS, aspirin, acetaminophen and/or codeine; or combinations of two or more thereof. For example, the desensitizing agent(s) may be chosen from potassium nitrate, potassium citrate, potassium chloride, potassium bicarbonate, potassium oxalate, and a combination of two or more thereof. In some embodiments, the desensitizing agent is potassium nitrate.

The oral care compositions may include one or more polyol(s). The one or more polyol(s) may be present in the oral care composition in an amount from about 1 to about 50 wt. %, based on the total weight of the oral care composition. For example, the oral care composition may include polyol(s) in an amount from about 1 to about 40 wt. %, about 1 to about 30 wt. %, about 1 to about 20 wt. %, about 1 to about 17 wt. %, about 1 to about 14 wt. %, about 1 to about 11 wt. %, about 1 to about 9 wt. %, about 1 to about 7 wt. %, about 1 to about 5 wt. %, about 1 to about 3 wt. %; from about 3 to about 50 wt. %, about 3 to about 40 wt. %, about 3 to about 30 wt. %, about 3 to about 20 wt. %, about 3 to about 17 wt. %, about 3 to about 14 wt. %, about 3 to about 11 wt. %, about 3 to about 9 wt. %, about 3 to about 7 wt. %; from about 6 to about 50 wt. %, about 6 to about 40 wt. %, about 6 to about 30 wt. %, about 6 to about 20 wt. %, about 6 to about 17 wt. %, about 6 to about 14 wt. %, about 6 to about 11 wt. %; from about 10 to about 50 wt. %, about 10 to about 45 wt. %, about 10 to about 40 wt. %, about 10 to about 35 wt. %, about 10 to about 30 wt. %, about 10 to about 25 wt. %, about 10 to about 20 wt. %, about 10 to about 17 wt. %, about 10 to about 14 wt. %; from about 15 to about 50 wt. %, about 15 to about 45 wt. %, about 15 to about 40 wt. %, about 15 to about 35 wt. %, about 15 to about 30 wt. %, about 15 to about 25 wt. %, about 15 to about 20 wt. %; from about 20 to about 50 wt. %, about 20 to about 45 wt. %, about 20 to about 40 wt. %, about 20 to about 35 wt. %, about 20 to about 30 wt. %, about 20 to about 25 wt. %; from about 30 to about 50 wt. %, about 30 to about 45 wt. %, about 30 to about 40 wt. %; from about 40 to about 50 wt. %, about 40 to about 45 wt. %, or any range or subrange thereof, based on the total weight of the oral care composition.

The polyol(s) may be chosen from glycols or compounds with numerous hydroxyl groups. The one or more polyols may be liquid at ambient temperature (25° C.). The polyol may be a humectant chosen from glycol, sorbitol, glycerin, inositol, maltitol, mannitol, xylitol, propylene glycol, polypropylene glycol (PPG), polyethylene glycol (PEG), a saccharide, polyoxypropylene-polyoxyethylene block copolymer and a combination of two or more thereof.

The polyol(s) may be selected from glycerin, glycol, inositol, maltitol, mannitol, sorbitol, xylitol, propylene glycol, polypropylene glycol (PPG), polyethylene glycol (PEG), a block copolymer of PPG and PEG, a saccharide (e.g., fructose, glucose, sucrose and mixtures of saccharides, such as honey), or a combination of two or more thereof. For instance, the oral care composition may comprise maltitol, mannitol, sorbitol, xylitol, a polypropylene glycol (PPG), a polyethylene glycol (PEG), a block copolymer of PPG and PEG, or a combination or two or more thereof.

In some cases, the oral care composition includes one or more polyol selected from the group consisting of C₂-C₃₂ polyols. The one or more polyols may have from 2 to 32 carbon atoms, from 3 to 16 carbon atoms, or from 3 to 12 carbon atoms. For example, the oral care composition may comprise ethylene glycol, propylene glycol, butylene glycol, hexylene glycol, glycerin, diglycerin, diethylene glycol, and dipropylene glycol, or a combination of two or more thereof. Additional, non-limiting examples of polyols that may, optionally, be incorporated in the oral care composition include alkanediols, such as glycerin, 1,2,6-hexanetriol, trimethylolpropane, ethylene glycol, propylene glycol, butylene glycol, pentylene glycol, hexylene glycol, diethylene glycol, triethylene glycol, tetraethylene glycol, pentaethylene glycol, dipropylene glycol, 2-butene-1,4-diol, 2-ethyl-1,3-hexanediol, 2-methyl-2,4-pentanediol, caprylyl glycol, 1,2-hexanediol, 1,2-pentanediol, and 4-methyl-1,2-pentanediol; glycol ethers, such as ethylene glycol monomethyl ether, ethylene glycol monoethyl ether, ethylene glycol monobutyl ether, ethylene glycol monomethyl ether acetate, diethylene glycol monomethyl ether, diethylene glycol monoethyl ether, diethylene glycol mono-n-propyl ether, ethylene glycol mono-iso-propyl ether, diethylene glycol mono-iso-propyl ether, ethylene glycol mono-n-butyl ether, ethylene glycol mono-t-butyl ether, diethylene glycol mono-t-butyl ether, 1-methyl-1-methoxybutanol, propylene glycol monomethyl ether, propylene glycol monoethyl ether, propylene glycol mono-t-butyl ether, propylene glycol mono-n-propyl ether, propylene glycol mono-iso-propyl ether, dipropylene glycol monomethyl ether, dipropylene glycol monoethyl ether, dipropylene glycol mono-n-propyl ether, dipropylene glycol mono-iso-propyl ether, sorbitol, sorbitan, triacetin, and a mixture thereof.

Additionally or alternatively, the oral care composition may include polyol(s) having a molecular weight of from about 100 to 5000 g/mol. For instance, the polyol may comprise a polyethylene glycol, a polypropylene glycol, a block polymer of polyethylene glycol and polypropylene glycol, or a combination of two or more thereof. In some embodiments, the polyol comprises a polypropylene glycol, a polypropylene glycol, and/or a block polymer of polyethylene glycol and polypropylene glycol having a molecular weight of about 100 to about 900, about 200 to about 800, about 400, about 1500 to about 2500, about 2000 to about 4500 or any range or subrange thereof. In some embodiments, the polyol is a polyethylene glycol, such as polyethylene glycol 600 (CAS-25322-68-3). In some embodiments, the oral care compositions comprise one or more polyethylene glycols, e.g., polyethylene glycols in a molecular weight range from 200 to 800. For example, the oral care composition may comprise polyethylene glycol 200, polyethylene glycol 300, polyethylene glycol 400, polyethylene glycol 600, polyethylene glycol 800, or a combination of two or more thereof.

In some embodiments, the oral care compositions may include one or more sweeteners. The oral care composition may include caloric sweeteners and/or non-caloric sweeteners. Examples of non-caloric sweeteners include saccharin, e.g., sodium saccharin, acesulfame, neotame, cyclamate or sucralose; natural high-intensity sweeteners, such as thaumatin, stevioside or glycyrrhizin; or sugar alcohols, such as sorbitol, xylitol, maltitol and mannitol. Examples of caloric sweeteners include sugars, such as fructose, glucose, sucrose, and high fructose syrups.

The one or more sweetener(s) may be present in the oral care composition in an amount from about 0.1 to about 7 wt. %, based on the total weight of the oral care composition. For example, the oral care composition may have a total amount of sweetener(s) from about 0.1 to about 5 wt. %, about 0.1 to about 4 wt. %, about 0.1 to about 3 wt. %, about 0.1 to about 2 wt. %, about 0.1 to about 1 wt. %; from about 0.5 to about 7 wt. %, about 0.5 to about 5 wt. %, about 0.5 to about 4 wt. %, about 0.5 to about 3 wt. %, about 0.5 to about 2 wt. %, about 0.5 to about 1 wt. %; from about 1 to about 7 wt. %, about 1 to about 5 wt. %, about 1 to about 4 wt. %, about 1 to about 3 wt. %, about 1 to about 2 wt. %; from about 2 to about 7 wt. %, about 2 to about 5 wt. %, about 2 to about 4 wt. %, about 2 to about 3 wt. %; from about 3 to about 7 wt. %, about 3 to about 5 wt. %, about 3 to about 4 wt. %; from about 4 to about 7 wt. %, about 4 to about 5 wt. %; from about 5 to about 7 wt. %, or any range or subrange thereof, based on the total weight of the oral care composition.

In some embodiments, the oral care composition preferably is substantially free or free of caloric sweeteners. For example, the oral care compositions may have about 4 wt. % or less, about 3 wt. % or less, about 2 wt. % or less, about 1 wt. % or less, about 0.5 wt. % or less, or about 0.1 wt. % or less, based on the weight of the oral care composition. In at least one embodiment, the oral care composition contains about 0 wt. % or 0 wt. % of caloric sweeteners, based on the weight of the oral care composition.

The oral care compositions of the present disclosure may include a flavoring agent. The flavoring agent is typically incorporated in the oral care composition at a concentration of about 0.01 to about 3 wt. % by weight of the oral care composition. For example, the amount of flavoring agent(s) present in the oral care composition may be from about 0.01 to about 2 wt. %, about 0.01 to about 1 wt. %, about 0.01 to about 0.5 wt. %, about 0.01 to about 0.1 wt. %; from about 0.1 to about 3 wt. %, about 0.1 to about 2 wt. %, about 0.1 to about 1 wt. %, about 0.1 to about 0.5 wt. %; from about 0.5 to about 3 wt. %, about 0.5 to about 2 wt. %, about 0.5 to about 1 wt. %; from about 1 to about 3 wt. %, about 1 to about 2 wt. %; from about 2 to about 3 wt. %, including any range or subrange thereof, based on the total weight of the oral care composition.

Suitable flavoring agents include, but are not limited to, essential oils and various flavoring aldehydes, esters, alcohols, and similar materials. Examples of the essential oils include oils of spearmint, peppermint, wintergreen, sassafras, clove, sage, eucalyptus, marjoram, cinnamon, lemon, lime, grapefruit, and orange. Also useful are such chemicals as menthol, carvone, and anethole. Additional flavoring agents may include, but are not limited to menthol, artificial vanilla, cinnamon derivatives, various fruit flavors, spearmint oil, peppermint oil, cinnamon oil, oil of wintergreen (methylsalicylate), clove oil, bay oil, anise oil, eucalyptus oil, thyme oil, cedar leaf oil, oil of nutmeg, oil of sage, oil of bitter almonds, cassia oil, and a combination of two or more thereof.

The oral care compositions may include one or more colorants. Exemplary colorants can include natural or uncertified colors from natural sources or certified colors for the effect of color. In some embodiments, the colorant can include dyes, certified aluminum lakes or colors derived from a natural source. The colorant may be water-based, oil-based or dry. The colorants can be primary colors, blends of colors or discrete mixtures of colors, such as confetti. The concentrations of the colorant in the oral care composition may be from trace amount to about 0.6 wt. %, from about 0.1 to about 0.5 wt. %, about 0.2 to about 0.4 wt. %, or about 0.15 to about 0.35 wt. %, based on the total weight of the oral care composition.

The oral care composition may include one or more pH adjusters to increase or decrease the overall pH of the oral care composition. For example, one or more acids may be included to decrease the pH of the oral care composition. Examples of suitable acids for decreasing the pH of the oral care composition include, but are not limited to, citric acid, acetic acid, and the like. The oral care composition may include one or more bases, such as sodium hydroxide, potassium hydroxide and the like, to increase the pH of the oral care composition. Additional or alternative acids and bases that are suitable for adjusting the pH of the oral care composition are readily known to one of ordinary skill in the art.

The amount of the pH adjuster in the oral care composition may be based on the desired pH of the final oral care composition and/or product. For example, the total amount of the pH adjuster may range from about 0.05 to about 20 wt. %, based on the total weight of the oral care composition. In some instances, the total amount of pH adjuster is from about 0.05 to about 15 wt. %, about 0.1 to about 10 wt. %, or about 0.12 to about 5 wt. %, including ranges and sub-ranges thereof, based on the total weight of the oral care composition.

The oral care composition may comprise from about 5 to about 90 wt. % of water, based on the total weight of the oral care composition. In some embodiment, the amount of water in the oral care composition is from about 5 to about 90 wt. %, about 5 to about 80 wt. %, about 5 to about 75 wt. %, about 5 to about 70 wt. %, about 5 to about 67 wt. %, about 5 to about 64 wt. %, about 5 to about 61 wt. %, about 5 to about 58 wt. %, about 5 to about 55 wt. %, about 5 to about 53 wt. %, about 5 to about 51 wt. %, about 5 to about 48 wt. %, about 5 to about 45 wt. %, about 5 to about 40 wt. %; from about 10 to about 90 wt. %, about 10 to about 80 wt. %, about 10 to about 75 wt. %, about 10 to about 70 wt. %, about 10 to about 67 wt. %, about 10 to about 64 wt. %, about 10 to about 61 wt. %, about 10 to about 58 wt. %, about 10 to about 55 wt. %, about 10 to about 53 wt. %, about 10 to about 51 wt. %, about 10 to about 48 wt. %, about 10 to about 45 wt. %, about 10 to about 40 wt. %; from about 20 to about 90 wt. %, about 20 to about 80 wt. %, about 20 to about 75 wt. %, about 20 to about 70 wt. %, about 20 to about 67 wt. %, about 20 to about 64 wt. %, about 20 to about 61 wt. %, about 20 to about 58 wt. %, about 20 to about 55 wt. %, about 20 to about 53 wt. %, about 20 to about 51 wt. %, about 20 to about 48 wt. %, about 20 to about 45 wt. %, about 20 to about 40 wt. %; from about 30 to about 90 wt. %, about 30 to about 80 wt. %, about 30 to about 75 wt. %, about 30 to about 70 wt. %, about 30 to about 67 wt. %, about 30 to about 64 wt. %, about 30 to about 61 wt. %, about 30 to about 58 wt. %, about 30 to about 55 wt. %, about 30 to about 53 wt. %, about 30 to about 51 wt. %, about 30 to about 48 wt. %, about 30 to about 45 wt. %; from about 40 to about 90 wt. %, about 40 to about 80 wt. %, about 40 to about 75 wt. %, about 40 to about 70 wt. %, about 40 to about 67 wt. %, about 40 to about 64 wt. %, about 40 to about 61 wt. %, about 40 to about 58 wt. %, about 40 to about 55 wt. %; from about 45 to about 90 wt. %, about 45 to about 80 wt. %, about 45 to about 75 wt. %, about 45 to about 70 wt. %, about 45 to about 67 wt. %, about 45 to about 64 wt. %, about 45 to about 61 wt. %; from about 50 to about 90 wt. %, about 50 to about 80 wt. %, about 50 to about 75 wt. %, about 50 to about 70 wt. %, about 50 to about 67 wt. %, about 50 to about 64 wt. %, about 50 to about 61 wt. %, about 50 to about 58 wt. %, about 50 to about 55 wt. %; from about 55 to about 90 wt. %, about 55 to about 80 wt. %, about 55 to about 75 wt. %, about 55 to about 70 wt. %, about 55 to about 67 wt. %, about 55 to about 64 wt. %, about 55 to about 61 wt. %; from about 60 to about 90 wt. %, about 60 to about 80 wt. %, about 60 to about 75 wt. %, about 60 to about 70 wt. %; from about 10 to about 90 wt. %, about 10 to about 80 wt. %, about 10 to about 75 wt. %, about 10 to about 70 wt. %, about 10 to about 67 wt. %, about 10 to about 64 wt. %, about 10 to about 61 wt. %, about 10 to about 58 wt. %, about 10 to about 55 wt. %, about 10 to about 53 wt. %, about 10 to about 51 wt. %, about 10 to about 48 wt. %, about 10 to about 45 wt. %, about 10 to about 40 wt. %; from about 70 to about 90 wt. %, about 70 to about 80 wt. %, about 80 to about 90 wt. %, or any range or subrange thereof, based on the total weight of the oral care composition.

In another aspect, the present disclosure provides a method of using the oral care compositions disclosed herein. The methods typically comprise applying any of the compositions as described herein to the teeth, e.g., by brushing, rubbing, swishing when the oral care composition is in the form of a dentifrice, or otherwise administering the oral care compositions to the oral cavity of a subject. The oral care compositions can be administered regularly, such as, for example, one or more times per day.

### EXAMPLES

### Example 1

Two example oral care compositions (Example Compositions A and B) were prepared in accordance with aspects of the invention. Five comparative oral care compositions (Comparative Compositions 1-5) were prepared using similar procedures as Example Compositions A and B. Example Compositions A and B and Comparative Compositions 1-5 were all in the form of a dentifrice and had formulations as presented in Table 1.

TABLE 1

| | Ex. A | Ex. B | Comp. 1 | Comp. 2 | Comp. 3 | Comp. 4 | Comp. 5 |
|---|---|---|---|---|---|---|---|
| US INCI Name | (wt. %) | (wt. %) | (wt. %) | (wt. %) | (wt. %) | (wt. %) | (wt. %) |
| Water | 53 | 53 | 51 | 55 | 52 | 56 | 59 |
| Propylene Glycol | 10 | 10 | 10 | 10 | 10 | 10 | 10 |
| Sodium Acid Pyrophosphate | 1.5 | 1 | 3 | | 2.7 | 2 | |
| Tetrasodium Pyrophosphate | 1 | 0.8 | 1 | 3 | 1 | 2 | |
| Sodium | 0.8 | 0.8 | 0.8 | 0.8 | 0.8 | 0.8 | 0.8 |
| Monofluorophosphate | | | | | | | |
| Sodium Saccharin | 0.6 | 0.6 | 0.6 | 0.6 | 0.6 | 0.6 | 0.6 |
| Sucralose | 0.05 | 0.05 | 0.05 | 0.05 | 0.05 | 0.05 | 0.05 |
| Hydrogen Peroxide | 3 | 3 | 3 | 3 | 3 | 3 | 3 |
| Calcium Pyrophosphate | 15 | 15 | 15 | 15 | 15 | 15 | 15 |
| Sodium Lauryl Sulfate | 2 | 1.5 | 2 | 2 | 2 | 2 | 2 |
| Powder | | | | | | | |
| Cool Flavor | 1.8 | 1.8 | 1.8 | 1.8 | 1.8 | 1.8 | 1.8 |
| Butylated Hydroxytoluene, | 0.03 | 0.03 | 0.03 | 0.03 | 0.03 | 0.03 | 0.03 |
| Fumed Silica | 3 | 3 | 5 | 2 | 3.5 | | 0 |
| Polyvinyl Pyrrolidone | 5 | 4 | 4 | 4 | 4 | 4 | 4 |
| Xanthan Gum | 0.5 | | | | | | |
| Faty alcohol and sodium alkyl | | 5 | | | | | |
| sulfate (Lanette) | | | | | | | |
| Sodium Stearate | | | | 2.5 | | | |
| Calcium Stearate | | | | | 4 | | |
| Stearic Acid | | | | | | 3 | |
| Carbopol | | | | | | | 1.5 |
| 50% KOH | | | | | | | 1.8 |

### Example 2

Example Compositions A and B and Comparative Compositions 1-5 were evaluated to assess the stability of such compositions. Specifically, each of the compositions was sealed in a PBL recyclable tube and kept in a temperature and humidity controlled environment at a temperature of 40° C. and relative humidity of 75% for 3 months. Example Compositions A and B and Comparative Compositions 1-5 were each evaluated before being placed in the temperature controlled environment, at one month of being in the temperature controlled, at two months of being in the temperature controlled environment, and at three months of being in the temperature controlled environment. The evaluation of Example Compositions A and B and Comparative Compositions 1-5 included determining the amount of active oxygen, the pH, and the physical stability of the compositions, and the amount of bloat exhibited by the sample. The level of bloat was evaluated on a scale from 0 to 5, with 0 being no bloating and 5 being a significant amount of bloating. When samples had unacceptable rheological properties (e.g., unacceptable texture, viscosity, and/or the composition could not stand up after dispensing), exhibited phase separation, or the samples exhibited a bloating of greater than 3, the aging study was not be conducted or continued.

Table 2 provides a summary of the stability of Example Compositions A and B and Comparative Compositions 1-5.

TABLE 2

| | Ex. A | | | Ex. B | | |
|---|---|---|---|---|---|---|
| | Active | | Physical | Active | | Physical |
| | Oxygen | pH | stability | Oxygen | pH | stability |
| Initial | 3.4% | 6.39 | Good initial | 3.1% | 6.51 | Good initial |
| | | | structure | | | structure |
| 1 month at 40° C. | 3.5% | 6.31 | No bloating | 3.0% | 6.1 | No bloating |
| 2 month at 40° C. | 3.4% | 6.21 | No bloating | 2.8% | 6.32 | Bloat 1 |
| 3 month at 40° C. | 3.1% | 6.27 | Bloat 2 | 2.8% | 6.14 | Bloat 2 |

| | Comp. 1 | | | Comp. 2 | | | Comp. 3 | | |
|---|---|---|---|---|---|---|---|---|---|
| | Active | | Physical | Active | | Physical | Active | | Physical |
| | Oxygen | pH | stability | Oxygen | pH | stability | Oxygen | pH | stability |
| Initial | 3.1% | 5.93 | Thicken up | N/A | 8.77 | Initial | N/A | 6.35 | Fail initial |
| | | | overnight, | | | texture good | | | structure: |
| | | | stand up | | | | | | flowable |
| | | | well. | | | | | | texture |

| | | | | | |
|---|---|---|---|---|---|
| 1 month at 40° C. | 3.2% | 5.97 | No bloating | Failed in fast screening: tube | The initial texture is not good; |
| 2 month at 40° C. | 2.9% | 5.98 | No bloating | bursted at 60° C. in 1 day; and | and tube bloats at room |
| 3 month at 40° C. | 2.9% | 6.07 | Bloat 1 | tube bloats at room | temperature after 4 months. |

temperature after 1 month.

| | Comp. 4 | | | Comp. 5 | | |
|---|---|---|---|---|---|---|
| | Active | | | Active | | |
| | Oxygen | pH | Physical stability | Oxygen | pH | Physical stability |
| Initial | N/A | 6.53 | Fail: phase | 3.1% | 6.05 | Texture good, stood up well |
| | | | separation | | | |
| 1 month at 40° C. | Initial texture failed, | | | 3.1% | 5.94 | Bloat 2, texture aerated and |
| | no aging study | | | | | loose structure |
| 2 month at 40° C. | needed. | | | 2.9% | 5.92 | Bloat 3, texture aerated and |
| | | | | | | loose structure |
| 3 month at 40° C. | | | | 2.8% | 5.91 | tube crimp open, texture |
| | | | | | | aerated and loose structure |

### Example 3

Example Composition A was evaluated in comparison to a non-aqueous commercial benchmark composition (Comparative Composition 6). A list of ingredients for Comparative Composition 6 is provided in Table 3.

TABLE 3

| | Comp. 6 |
|---|---|
| US INCI Compound Name | (wt. %) |
| Propylene Glycol | 53 |
| Polyethylene Glycol/Polypropylene | 7.5 |
| Glycol 116/66 Copolymer | |
| Calcium Pyrophosphate | 15 |
| Fumed Silica | 0.4 |
| Tetrasodium Pyrophosphate | 1.3 |
| Sodium Acid Pyrophosphate | 0.6 |
| Sodium Monofluorophosphate | 0.8 |
| Cross-Linked PVP Complexed with | 16.5 |
| Hydrogen Peroxide | |
| Sodium Saccharin Powder | 0.8 |
| Sucralose | 0.05 |
| Flavor | 2.3 |
| Butylated Hydroxytoluene | 0.03 |
| Sodium Lauryl Sulfate Powder | 2 |

Specifically, Example Composition A and Comparative Composition 6 were evaluated using an in vitro brushing protocol using stained bovine teeth. The artificially stained bovine central incisors (stained with coffee and tea broth), which were mounted in a resin, were obtained from Dental Product Testing Therametric Technologies, Inc.

The teeth were pre-brushed to have a starting L* value between 58 and 62. A 10 gram sample of Example Composition A and a 10 gram sample of Comparative Composition 6 were separately dispersed in 10 grams of distilled water to produce a solution containing Example Composition A and a solution containing Comparative Composition 6. The solutions were then used for brushing the bovine incisors for 2 minutes under 250 grams of force at 120 strokes/min.

The L*, a*, b* values of the bovine incisors were measured using a hyperspectral camera, available from Middleton Spectral Vision. The L* a*, b* values were measured after every two brushings, and 14 brushings were conducted to evaluate a whitening performance corresponding with 7 days of brushing an individual's teeth using such dentifrices. The whitening performance was then calculated as the change in W value after treatments compared to baseline value. The equations used to calculate ΔW from L*, a*, b* values are shown below. Absolute ΔW values were used to have positive values. The higher the absolute ΔW value, the whiter the tooth compared to the baseline measurement.

$${W}^{*} = {( {a}^{* 2} + {b}^{* 2} + {( {L}^{*} - 100 )}^{2} )}^{1 / 2} \\ Δ W = {W}_{treated}^{*} - {W}_{baseline}^{*}$$

As seen in Table 4, below, Example Composition A provided a comparable whitening efficacy as the non-aqueous commercial benchmark composition (Comparative Composition 6). Achieving a comparable whitening effect with non-aqueous Comparative Composition 6 was a surprisingly good result as aqueous oral care compositions utilizing hydrogen peroxide as the whitening agent often provide inferior whitening effects to non-aqueous oral care compositions utilizing hydrogen peroxide as the whitening agent due to the increased degradation of hydrogen peroxide in aqueous solutions.

TABLE 4

| | Absolute ΔW | Absolute ΔW |
|---|---|---|
| Time Point | of Ex. A | of Comp. 6 |
| Baseline | 0 | 0 |
| Day 1 | 3.93 | 3.51 |
| Day 2 | 5.03 | 5.08 |
| Day 3 | 6.28 | 6.04 |
| Day 4 | 6.99 | 7.01 |
| Day 5 | 7.73 | 7.78 |
| Day 6 | 8.68 | 8.32 |
| Day 7 | 8.67 | 8.72 |

### Example 4

Six non-limiting example compositions (Example Compositions C-H) were prepared in accordance with aspects of the invention. Example Compositions C-H were in the form of a dentifrice and had formulations as shown in Table 5. The formulation of Example Composition A is reproduced in Table 5.

TABLE 5

| | Ex. A | Ex. C. | Ex. D | Ex. E | Ex. F | Ex. G | Ex. H |
|---|---|---|---|---|---|---|---|
| US INCI Name | (wt. %) | (wt. %) | (wt. %) | (wt. %) | (wt. %) | (wt. %) | (wt. %) |
| Propylene Glycol | 10 | 10 | 10 | 10 | 10 | 10 | 10 |
| Sodium Acid Pyrophosphate | 1.5 | 1.5 | 1.5 | 1.5 | 1.5 | 1.5 | 1.5 |
| Tetrasodium Pyrophosphate | 1 | 1 | 1 | 1 | 1 | 1 | 1 |
| Sodium | 0.76 | 0.76 | 0.76 | 0.76 | 0.76 | 0.76 | 0.76 |
| Monofluorophosphate | | | | | | | |
| Sodium Saccharin | 0.6 | 0.6 | 0.6 | 0.6 | 0.6 | 0.6 | 0.6 |
| Sucralose | 0.05 | 0.05 | 0.05 | 0.05 | 0.05 | 0.05 | 0.05 |
| Hydrogen Peroxide | 3 | 3 | 3 | 3 | 3 | 3 | 3 |
| Calcium Pyrophosphate | 15 | 15 | 15 | 15 | 15 | 15 | 15 |
| Sodium Lauryl Sulfate | 2 | 2 | 2 | 2 | 2 | 2 | 2 |
| Powder | | | | | | | |
| Cool Flavor | 1.8 | 1.8 | 1.8 | 1.8 | 1.8 | 1.8 | 1.8 |
| Butylated Hydroxytoluene, | 0.03 | 0.03 | 0.03 | 0.03 | 0.03 | 0.03 | 0.03 |
| Fumed Silica | 3 | | 1 | 1.25 | 5 | 3.3 | 3 |
| Polyvinyl Pyrrolidone | 5 | 7 | 9 | 9 | 4 | | 3 |
| Xanthan Gum | 0.5 | 0.8 | 0.5 | 0.35 | 0.25 | 0.9 | 0.7 |
| Water | Q.S. to | Q.S. to | Q.S. to | Q.S. to | Q.S. to | Q.S. to | Q.S. to |
| | 100 | 100 | 100 | 100 | 100 | 100 | 100 |

### Example 5

Example Compositions A and C-H were evaluated to assess the stability of such compositions according to the procedures similar to those described in Example 2. Each of the compositions was sealed in a PBL recyclable tube and kept in a temperature and humidity controlled environment at a temperature of 40° C. and relative humidity of 75% for 13 weeks. Example Compositions A and C-H were evaluated before being placed in the temperature and humidity controlled environment and at 13 weeks of remaining in the temperature and humidity controlled environment.

The initial texture, active oxygen, and level of tube bloat was evaluated as summarized in Table 6. The level of bloat was evaluated on a scale from 0 to 5. with 0 being no bloating and 5 being a significant amount of bloating.

TABLE 6

| | | | Level |
|---|---|---|---|
| | | Δ Peroxide(Initial | of Tube |
| | Initial Texture | to Final wt. %) | Bloat |
| Example Composition A | Good Texture | 3.3% to 2.9% | 2 |
| Example Composition C | Good Texture | 3.2% to 2.9% | 2 |
| Example Composition D | Good Texture | 3.2% to 2.7% | 2 |
| Example Composition E | Good Texture | 3.3% to 3.0% | 2 |
| Example Composition F | Good Texture | 3.1% to 2.95% | 2 |
| Example Composition G | Good Texture | 3.07% to 2.83% | 2 |
| Example Composition H | Good Texture | 2.98% to 2.96% | 2 |

1-33. (canceled)

34. An oral care composition comprising:
a whitening agent comprising from a peroxide;
a thickening system comprising two or more of:
(i) a silica thickener,
(ii) crosslinked polyvinyl pyrrolidone, or
(iii) one of xanthan gum, cetyl alcohol, or a combination thereof;
an abrasive comprising an insoluble phosphate salt; and
from about 5 to 90 wt. % of water,
wherein the oral care composition has a pH of about 4 to about 8, and all weight percentages are based on the total weight of the oral care composition.

35. The oral care composition according to claim 34, wherein the whitening agent is selected from hydrogen peroxide, lithium peroxide, potassium peroxide, sodium peroxide, magnesium peroxide, calcium peroxide, barium peroxide, alkyl hydrogen peroxides, dialkyl peroxides, alkyl peroxy acids, peroxy esters, diacyl peroxides, benzoyl peroxide, and monoperoxyphthalate, cross-linked PVP complexed with hydrogen peroxide, silica complexed with hydrogen peroxide and a combination of two or more thereof.

36. The oral care composition according to claim 34, wherein the peroxide is a non-complexed peroxide present in the amount of from about 0.01 to about 10 wt. %.

37. The oral care composition according to claim 34, wherein the thickening system comprises xanthan gum.

38. The oral care composition according to claim 34, wherein the xanthan gum is present in an amount from about 0.1 to about 10 wt. %.

39. The oral care composition according to claim 34, wherein the thickening system comprises cetyl alcohol, and wherein the oral care composition further comprises an alkyl sulfate surfactant.

40. The oral care composition according to claim 34, wherein the thickening system is present in an amount from about 0.5 to about 20 wt. %.

41. The oral care composition according to claim 34, wherein the silica thickener is selected from a fumed silica.

42. The oral care composition according to claim 34, wherein the silica thickener is present in an amount from about 0.5 to about 10 wt. %.

43. The oral care composition according to claim 34, wherein the crosslinked polyvinyl pyrrolidone is present in an amount from about 0.5 to about 20 wt. %.

44. The oral care composition according to claim 34, wherein insoluble phosphate salt comprises calcium pyrophosphate, the calcium pyrophosphate comprising about 60 wt. % or more of the total amount of the abrasive in the oral care composition.

45. The oral care composition according to claim 34 further comprising:
a humectant in an amount from about 1 to about 50 wt. %.

46. The oral care composition according to claim 45, wherein the humectant comprises a glycol, sorbitol, glycerin, inositol, maltitol, mannitol, xylitol, propylene glycol, polypropylene glycol (PPG), polyethylene glycol (PEG), a saccharide, Polyoxypropylene-Polyoxyethylene Block Copolymer and a combination of two or more thereof.

47. The oral care composition according to claim 34, wherein the amount of water is from about 40 to about 90 wt. %.

48. The oral care composition according to claim 34 further comprising: potassium nitrate in an amount from about 0.1 to about 9 wt. %.

49. The oral care composition according to claim 34, wherein the oral care composition has a loss of peroxide of about 30% or less over a 13-week period at 40° when sealed in a dentifrice PBL recyclable tube.

50. The oral care composition according to claim 34, wherein the oral care composition has a loss of peroxide of about 14% or less over a 13-week period at 40° when sealed in a dentifrice PBL recyclable tube.

51. The oral care composition according to claim 34, wherein the peroxide is selected from non-complexed peroxides.

52. An oral care composition comprising:
a whitening agent comprising a peroxide;
a thickening system comprising two or more of:
(i) a silica thickener,
(ii) crosslinked polyvinyl pyrrolidone, or
(iii) xanthan gum;
an abrasive comprising calcium pyrophosphate;
from about 1 to about 50 wt. % of a humectant; and
from about 5 to 90 wt. % of water,
wherein the oral care composition has a pH of about 4 to about 8, and all weight percentages are based on the total weight of the oral care composition.

53. An oral care composition comprising:
a whitening agent comprising a peroxide;
a thickening system comprising two or more of:
(i) a silica thickener,
(ii) crosslinked polyvinyl pyrrolidone, or
(iii) cetyl alcohol;
an anionic sulfate based surfactant;
an abrasive comprising calcium pyrophosphate;
from about 1 to about 50 wt. % of a humectant; and
from about 5 to 90 wt. % of water,
wherein the oral care composition has a pH of about 4 to about 8, and all weight percentages are based on the total weight of the oral care composition.
